(19) United States
(12) Patent Application Publication
(10) Pub. No.: US 2014/0142693 A1
(43) Pub. Date: May 22, 2014

(54) Valve Prosthesis Frames

(71) Applicant: MEDTRONIC, INC, Minneapolis, MN (US)

(72) Inventors: Michael Krivoruchko, Santa Rosa, CA (US); Karan Punga, Santa Rosa, CA (US); Finn Rinne, Santa Rosa, CA (US)

(73) Assignee: Medtronic, Inc, Minneapolis, MN (US)

(21) Appl. No.: 13/681,594

(22) Filed: Nov. 20, 2012

(51) Int. Cl. A61F 2/24

(52) U.S. Cl. CPC A61F 2/2409
USPC 623/2.18

(57) ABSTRACT

A prosthesis can include a collapsible, reexpandable frame comprising first, second, and third sets of struts that define first and second rows of expandable cells. In some embodiments, the struts of the first, second, and third set of struts can be tapered. In some embodiments, the frame can include an intermediate section and an inflow section that is proximal to the intermediate section. The inflow section can include a concave saddle portion that is adjacent the intermediate section, and an outwardly flared portion.

### BACKGROUND

1. Field

Embodiments of the present invention relate to valve prostheses and, particularly, to frames for valve prostheses.

2. Background

Patients suffering from valve regurgitation or stenotic calcification of the leaflets can be treated with a heart valve replacement procedure. A traditional surgical valve replacement procedure requires a sternotomy and a cardiopulmonary bypass, which creates significant patient trauma and discomfort. Traditional surgical valve procedures can also require extensive recuperation times and may result in life-threatening complications.

One alternative to a traditional surgical valve replacement procedure is delivering the replacement heart valve prosthesis using minimally-invasive techniques. For example, a heart valve prosthesis can be percutaneously and transluminally delivered to an implantation location. In such methods, a heart valve prosthesis can be compressed to be loaded within a sheath of a delivery catheter or crimped on a delivery catheter; advanced to the implantation location; and re-expanded to be deployed at the implantation location. For example, a catheter loaded with a compressed heart valve prosthesis can be introduced through an opening in the femoral artery and advanced through the aorta to the heart. At the heart, the prosthesis can be re-expanded to be deployed at the aortic valve annulus, for example.

### BRIEF SUMMARY

In some embodiments, a prosthesis can include a collapsible, expandable frame that has a longitudinal axis. The frame can include first, second, and third sets of struts. The first and second sets of struts can be connected at a first plurality of nodes to define a first row of expandable cells. The second and third sets of struts can be connected at a second plurality of nodes to define a second row of expandable cells. The first row of expandable cells can be proximal to the second row of expandable cells. Each strut of the first set of struts can include a first proximal segment with a first width, a first intermediate segment with a second width, and a first distal segment with a third width. Each strut of the second set of struts can include a second proximal segment with a fourth width, a second intermediate segment with a fifth width, and a second distal segment with a sixth width. Each strut of the third set of struts can include a third proximal segment with a seventh width, a third intermediate segment with an eighth width, and a third distal segment with a ninth width. The second width can be smaller than each of the first and third widths. The fifth width can be smaller than each of the fourth and sixth widths. The eighth width can be smaller than each of the seventh and ninth widths.

In some embodiments, a prosthesis can include an expandable frame that has a longitudinal axis. The frame can include an intermediate section and an inflow section that is proximal to the intermediate section. The inflow section can include a concave saddle portion that is adjacent the intermediate section, and an outwardly flared portion.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS

The accompanying drawings, which are incorporated herein and form part of the specification, illustrate embodiments of the present invention and, together with the description, further serve to explain the principles of the invention and to enable a person skilled in the relevant art(s) to make and use the invention.

FIG. 1 schematically illustrates a profile of a valve prosthesis frame according to an embodiment.

FIG. 2 illustrates a valve prosthesis having a profile similar to the profile illustrated in FIG. 1.

FIG. 3 illustrates a valve prosthesis according to another embodiment.

FIG. 4 illustrates an enlarged view of first and second rows of expandable cells of a frame according to an embodiment.

FIG. 5 illustrates another enlarged view of first and second rows of expandable cells of a frame according to an embodiment.

FIG. 6 illustrates a graph showing the loading and unloading force curves of valve prostheses according to two embodiments.

FIG. 7 is an enlarged view of a portion of the graph illustrated in FIG. 6.

FIG. 8 illustrates an enlarged view of first and second rows of expandable cells of the frame illustrated in FIG. 2 in a compressed condition.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

This specification discloses embodiments that incorporate the features of this invention. The disclosed embodiments merely exemplify the invention. The scope of the invention is not limited to the disclosed embodiments. The invention is defined by the claims appended hereto.

The embodiments described, and references in the specification to “one embodiment,” “an embodiment,” “an example embodiment,” “some embodiments,” etc., indicate that the embodiments described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

In this application, the term “proximal” means situated nearer to the upstream or inflow side of the valve prosthesis, and the term “distal” means situated nearer to the downstream or outflow side of the valve prosthesis.

A valve prosthesis according to various embodiments can be used to replace the function of a native cardiac valve, for example, the tricuspid valve, the pulmonary valve, the mitral valve, and the aortic valve. In some embodiments, the heart valve prosthesis can be configured to be collapsed to a small diameter condition such that the valve prosthesis can be delivered into a patient's body using minimally invasive techniques. For example, the heart valve prosthesis can be collapsed to a small diameter condition that allows the valve prosthesis to be loaded within a delivery sheath of delivery catheter or crimped on the delivery catheter. In some embodiments, the valve prosthesis can be delivered using a catheter, a laparoscopic instrument, or any other suitable delivery device. In some embodiments, the valve prosthesis can be delivered to the heart using a transfemoral, transapical, transaxial, transaortic, or transseptal approach.

In some embodiments, the valve prosthesis can be configured to be re-expandable such that once the collapsed heart valve prosthesis is delivered to a desired implantation site the valve prosthesis can be deployed by re-expanding the valve prosthesis to a larger diameter condition to securely engage the surrounding anatomy. For example, an aortic valve prosthesis may be expanded to engage the aorta, the aortic annulus, the left ventricle outflow tract, or combinations thereof. After deployment, the valve prosthesis can function as a one-way valve, permitting blood flow in one direction (a downstream or distal direction) and preventing blood flow in an opposite direction (an upstream or proximal direction).

In some embodiments, the valve prosthesis can be configured to be fully or partially recaptured within a sheath of a delivery catheter. Such recapturing can occur during or after deployment at the implantation site. For example, after partially or fully deploying the valve prosthesis, a clinician may wish to reposition or remove the prosthesis. The clinician can recapture the valve prosthesis within a sheath of a catheter and reposition or remove the valve prosthesis.

In some embodiments, the valve prosthesis includes a frame and a valve assembly coupled thereto. The frame supports the valve assembly. In some embodiments, the frame can be configured to be self-expandable or balloon expandable. The frame can be made from any suitable biocompatible metal and synthetic material. For example, suitable biocompatible metals can include nickel, titanium, stainless steel, cobalt, chromium, alloys thereof (e.g., nitinol), or any other suitable metal. And for example, suitable biocompatible synthetic materials can include thermoplastics or any other suitable synthetic material.

In some embodiments, the valve assembly includes a plurality of leaflets. In some embodiments, the valve assembly can also include a skirt. The valve assembly can be made from any suitable synthetic or biological material. For example, suitable biological materials can include mammalian tissue such as porcine, equine, or bovine pericardium.

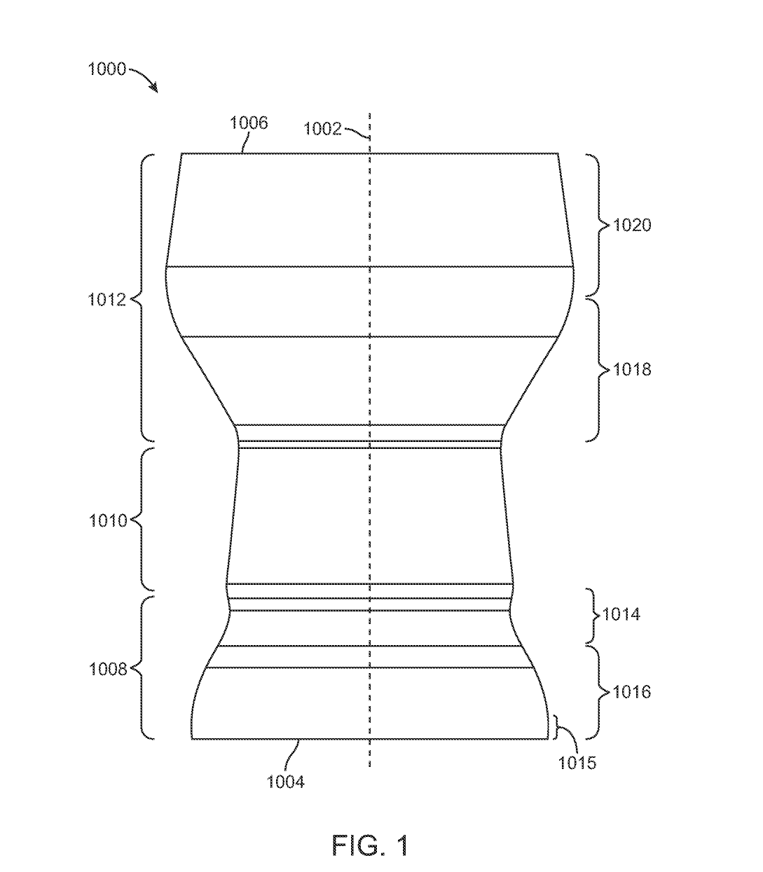

FIG. 1 schematically illustrates a profile of a prosthesis frame 1000 in an expanded condition according to an embodiment. Frame 1000 has an longitudinal axis 1002. Frame 1000 also includes an inflow edge 1004 and an outflow edge 1006.

Frame 1000 can have an inflow section 1008, an intermediate section 1010, and an outflow section 1012. In some embodiments, inflow section 1008 can include a saddle portion 1014 and an outwardly flared portion 1016. Saddle portion 1014 can be concave—curving or bending inward towards longitudinal axis 1002 as shown in FIG. 1. In some embodiments, the contour of saddle portion 1014 forms a smooth curve. In some embodiments, the diameter of saddle portion 1014 at an intermediate section is smaller than the diameter of saddle portion 1014 at its distal edge and at its proximal edge. Outwardly flared portion 1016 can include at least one portion that extends outwardly away from longitudinal axis 1002 as flared portion 1016 extends in a proximal direction. In some embodiments, outwardly flared portion 1016 is convex—curving or bending outward away from longitudinal axis 1002 as shown in FIG. 1. In some embodiments, the contour of outwardly flared portion 1016 forms a smooth convex curve. In some embodiments, the diameter of outwardly flared portion 1016 at an intermediate section is greater than the diameter of outwardly flared portion 1016 at its distal edge.

In some embodiments, a portion 1015 of outwardly flared portion 1016 at inflow edge 1004 extends towards longitudinal axis 1002 as shown in FIG. 1. In such embodiments, the diameter of outwardly flared portion 1016 at an intermediate section is greater than the diameter of outwardly flared portion 1016 at its inflow edge 1004. In some embodiments, inwardly tapered portion 1015 of outwardly flared portion 1016 can reduce tissue trauma and heart block.

As shown in FIG. 1, the transition between saddle portion 1014 and outwardly flared portion 1016 can form a smooth curve. In some embodiments, inflow section 1008 can have a length along longitudinal axis 1002 that ranges from about 2 mm to about 50 mm. For example, a longitudinal length of inflow section 1008 can range from about 10 mm to about 20 mm.

Intermediate section 1010 can be adjacent inflow section 1008. In some embodiments, intermediate section 1010 can be adjacent saddle portion 1014 of inflow section 1008. In some embodiments, the contour of intermediate section 1010 is generally parallel to longitudinal axis 1002. For example, the contour of intermediate section 1010 can taper slightly inward (as shown in FIG. 1) or outward as it extends in the distal direction, or can be exactly parallel to longitudinal axis 1002. In embodiments in which the contour of intermediate section 1010 tapers inward as it extends in the distal direction, the diameter of intermediate portion 1010 at its proximal edge is greater than the diameter of intermediate portion 1010 at its distal edge.

Outflow section 1012 can be adjacent intermediate section 1010. In some embodiments, outflow section 1012 includes a first portion 1018 that is adjacent the distal edge of intermediate section 1010, and a second portion 1020 that is adjacent first portion 1018. In some embodiments, first portion 1018 is outwardly flared—the contour surface of first portion 1018 extends away from longitudinal axis 1002 as first portion 1018 extends in the distal direction. In some embodiments, a second portion 1020 is inwardly flared—the contour surface of second portion 1020 extends towards longitudinal axis 1002 as second portion 1020 extends in the distal direction. In some embodiments, inwardly flared portion 1020 defines outflow edge 1006. In some embodiments, the transition between outwardly flared portion 1018 and inwardly flared portion 1020 forms a smooth curve.

In some embodiments, the diameter of outflow portion 1012 at an intermediate section, for example, the transition between first portion 1018 and second portion 1020, is greater than the diameter of outflow portion 1012 at its outflow edge 1006 and at its proximal edge.

In some embodiments, frame 1000 is configured to be implanted at the aortic valve. Accordingly, inflow section 1008 can be configured to have an expanded diameter such that inflow section 1008 engages the outflow tract of the left ventricle. Saddle portion 1014 can be configured to have an expanded diameter such that saddle portion 1014 engages the transition between the native aortic annulus and the left ventricle outflow tract. Intermediate section 1010 can be configured to have an expanded diameter such that intermediate section 1010 engages the native aortic annulus or leaflets. Second portion 1020 can be configured to have an expanded diameter such that second portion 1020 engages the aorta at a supra-coronary position. In some embodiments, the outwardly flared configuration of first portion 1018 of outflow section 1012 is configured to provide a radial gap between frame 1000 and the aorta. This radial gap can reduce the risk that the coronary arteries will be blocked.

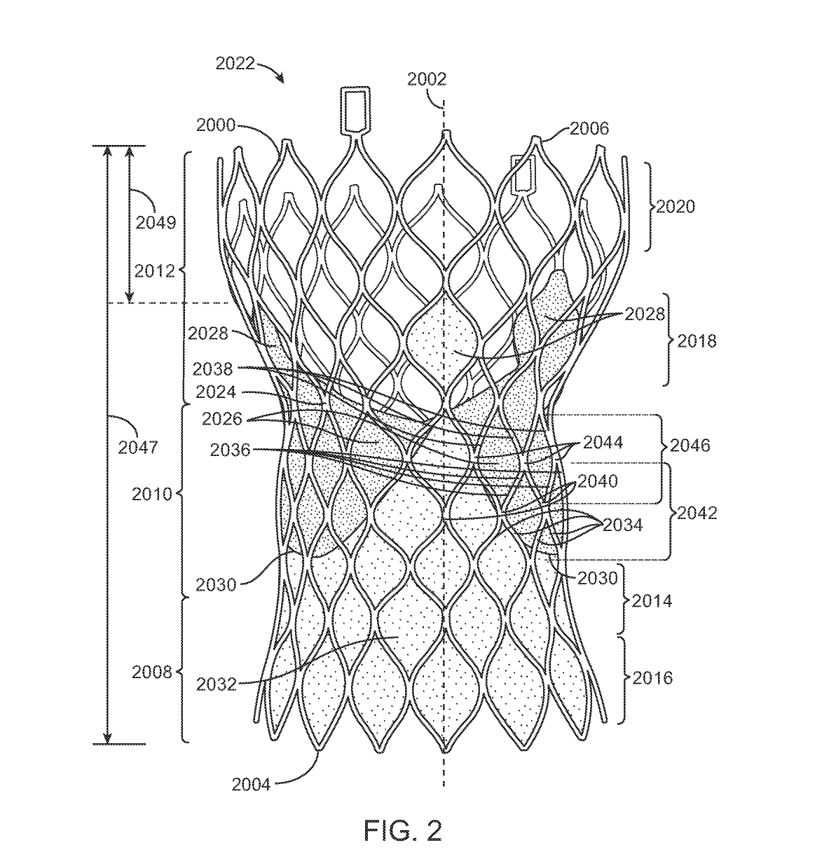

FIG. 2 illustrates a valve prosthesis 2022 that includes a frame 2000 and a valve assembly 2024 according to an embodiment. As shown in FIG. 2, frame 2000 has a profile similar to the profile of frame 1000 illustrated in FIG. 1. Accordingly, frame 2000 includes similar features as the above described frame 1000. These similar features are similarly numbered and function and can be configured generally the same as they are in frame 1000. Thus, these similar features of frame 2000 are generally only discussed to the extent they may differ from the embodiments described above with reference to FIG. 1 or to the extent necessary to describe features of valve prosthesis 2022.

In some embodiments, valve assembly 2024 includes a plurality of leaflets 2026. For example, valve assembly 2024 can include three leaflets 2026 (only two leaflets 2026 are shown in FIG. 2). Adjacent leaflets 2026 are attached to one another at their lateral sides to form commissures 2028. In some embodiments, valve assembly 2024 includes a skirt 2032. The cusps of leaflets 2026 can be coupled to skirt 2032 using any suitable attachment mechanism, for example, sutures or adhesive.

In some embodiments, frame 2000 comprises a plurality of struts forming expandable cells. For example, as shown in FIG. 2, frame 2000 includes at least a first set of struts 2034, a second set of struts 2036, and a third set of struts 2038. In some embodiments, each set of struts 2034, 2036, and 2038 can have an undulating pattern, for example, a sinusoidal pattern or a zigzag pattern. First set of struts 2034 can be coupled to second set of struts 2036 at a first plurality of nodes 2040 to form a first row of expandable cells 2042. Second set of struts 2036 can be coupled to third set of struts 2038 at a second plurality of nodes 2044 to form a second row of expandable cells 2046. As shown in FIG. 2, this strut pattern can be axially repeated to form a plurality of rows of expandable cells along an entire longitudinal length 2047 of valve prosthesis 2022.

In some embodiments, valve assembly 2024 is mounted within frame 2000 such that nadirs 2030 of leaflets 2026 are positioned adjacent to the distal edge of saddle portion 2014 of inflow section 2008.

In some embodiments, saddle portion 2014 comprises first and second sets of struts defining only a single row of expandable cells. In some embodiments, saddle portion 2014 comprises more than two sets of struts defining two or more rows of expandable cells.

In some embodiments, first portion 2018 of outflow section 2012 is outwardly flared such that the contour extends away from longitudinal axis 2002. In some embodiments, commissures 2028 are coupled to outwardly flared portion 2018 of outflow section 2012. In some embodiments, valve assembly 2024 is mounted within frame 2000 such that the valve assembly 2024 has a high profile valve position. For example, a longitudinal length 2049 between commissures 2028 and outflow edge 2006 is about 20% to about 35% of longitudinal length 2047 of frame 2000. A high-profile valve position can improve hemodynamics and durability.

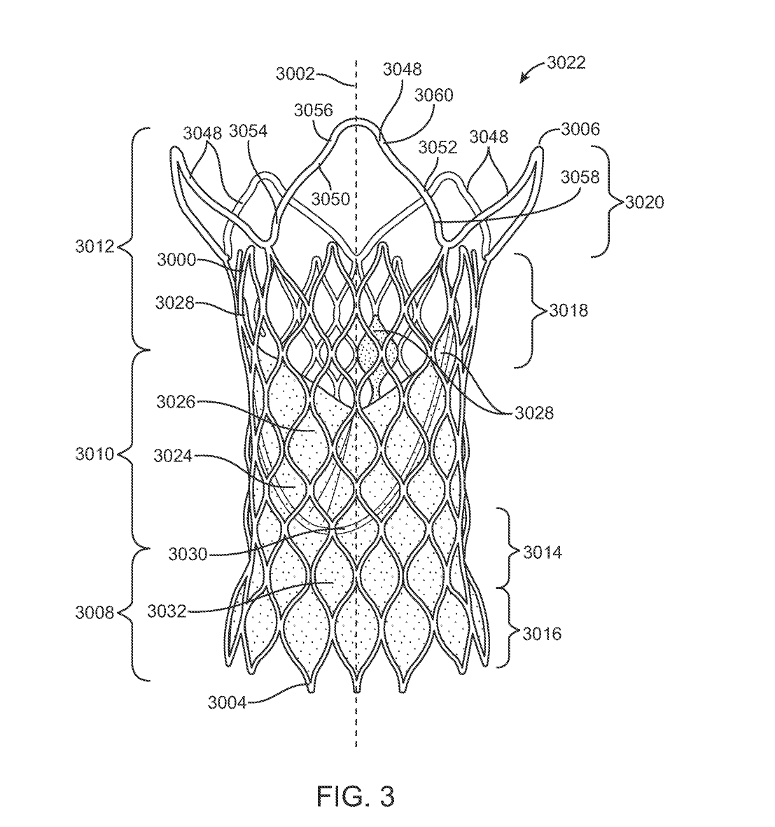

FIG. 3 illustrates a valve prosthesis 3022 according to another embodiment. Valve prosthesis 3022 includes a frame 3000 and a valve body 3024. Valve prosthesis 3022 includes similar features as the above described valve prosthesis 2022 and frame 1000. These similar features are similarly numbered and function and can be configured generally the same as they are in valve prosthesis 2022 and frame 1000. Thus, these similar features of valve prosthesis 3022 are generally only discussed to the extent they may differ from the embodiments described above with reference to FIGS. 1 and 2 or to the extent necessary to describe features of valve prosthesis 3022.

In some embodiments, frame 3000 includes an inflow section 3008, an intermediate section 3010, and an outflow section 3012. Outflow section 3012 can have a first portion 3018 that is adjacent intermediate portion 3010, and a second portion 3020. In some embodiments, first portion 3018 has a contour that is substantially parallel to longitudinal axis 3002. For example, the contour of first portion 3018 can taper slightly outward (as shown in FIG. 3) or inward as it extends in the distal direction, or can be exactly parallel to longitudinal axis 3002.

In some embodiments, second portion 3020 can include a plurality of V-shaped structures 3048. For example, second portion 3020 can include five V-shaped structures 3048 as shown in FIG. 3. Each V-shaped structure 3048 can include a first strut 3050 having a proximal end 3054 and a distal end 3056, and a second strut 3052 having a proximal end 3058 and a distal end 3060. Each distal end 3056 of first struts 3050 is joined to a distal end 3060 of an adjacent second strut 3052 to form an apex of V-shaped structure 3048. Each proximal end 3054 of first struts 3050 is joined to a node defining an expandable cell, and each proximal end 3058 is joined to a node defining an expandable cell spaced apart from the expandable cell joined to the respective proximal end 3054. In some embodiments, at least one cell is between the cell to which proximal end 3054 is attached and the cell to which proximal end 3058 is attached. For example, as shown in FIG. 3, two cells can be between the cell to which proximal end 3054 is attached and the cell to which proximal end 3058 is attached. In some embodiments, V-shaped structures 3048 are convex—curving or bending away from longitudinal axis 3002. V-shaped structures 3048 can improve loading and anatomical fit.

In some embodiments, commissures 3028 are coupled to first portion 3018 of outflow section 3012 that has a contour surface that is substantially parallel to longitudinal axis 3002. Attaching commissures 3028 in this manner can improve valve assembly durability.

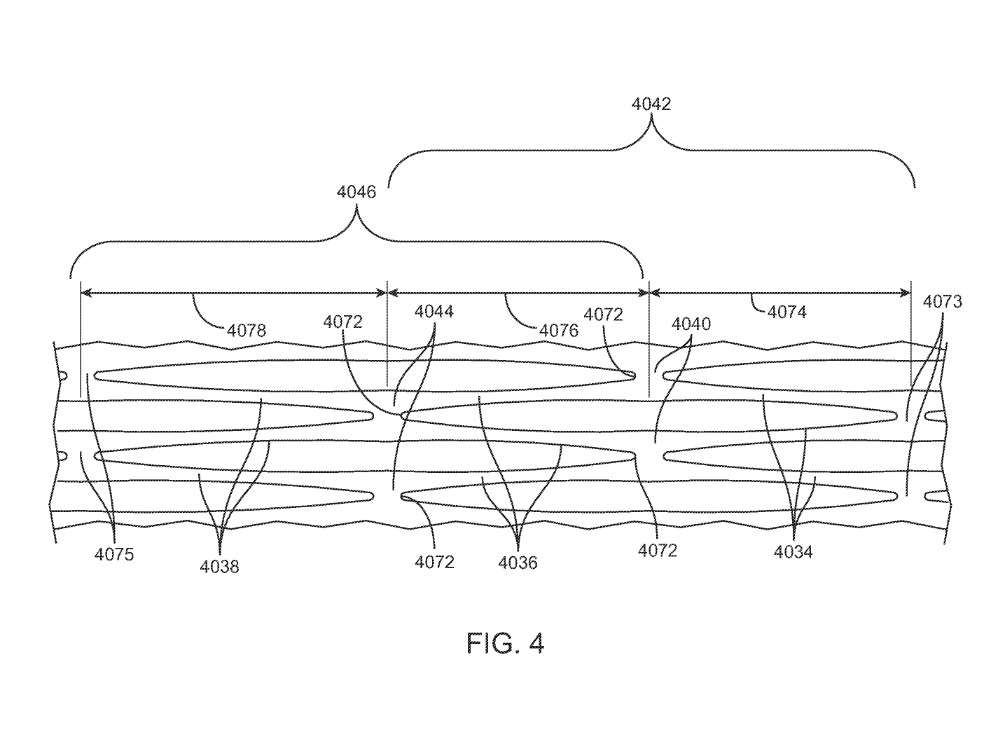

FIG. 4 illustrates an enlarged view of a first row 4042 and a second row 4046 of expandable cells of a frame in its original manufactured state—prior to being expanded or compressed—according to an embodiment. This cell pattern can be used in any section of frame 10000 illustrated in FIG. 1, frame 2000 illustrated in FIG. 2, or frame 3000 illustrated in FIG. 3. For example, this cell pattern can be used to form a portion of inflow section 2008, intermediate section 2010, or outflow section 2012 of frame 2000. In some embodiments, this cell pattern can form the entire intermediate section 2010 of frame 2000. Similarly, this cell pattern can be used to form a portion of inflow section 3008, intermediate section 3010, or outflow section 3012 of frame 3000. In some embodiments, this cell pattern can form the entire intermediate section 3010 of frame 3000.

As shown in FIG. 4, the frame can include a plurality of struts forming a first row 4042 and a second row 4046 of expandable cells. For example, the frame can include a first set of struts 4034, a second set of struts 4036, and a third set of struts 4038. In some embodiments, each set of struts 4034, 4036, and 4038 can have an undulating pattern, for example, a sinusoidal pattern or a zigzag pattern. In some embodiments to form first row of expandable cells 4042, distal ends of each strut of first set of struts 4034 can be coupled to proximal ends of respective struts of second set of struts 4036 at a first plurality of nodes 4040. In some embodiments to form second row of expandable cells 4046, distal ends of each strut of second set of struts 4036 can be coupled to proximal ends of respective struts of third set of struts 4038 at a second plurality of nodes 4044. In some embodiments, proximal ends of each strut of first set of struts 4034 can be coupled to adjacent proximal struts (not shown in FIG. 4) at a third plurality of nodes 4073. In some embodiments, distal ends of each strut of third set of struts 4038 are coupled to adjacent distal struts (not shown in FIG. 4) at a fourth plurality of nodes 4075.

In some embodiments, a longitudinal length 4074 between a center of third plurality of nodes 4073 and first plurality of nodes 4040 is about equal to a longitudinal length 4076 between a center of first plurality of nodes 4040 and a center of second plurality of nodes 4044. In some embodiments, longitudinal lengths 4074 and 4076 are within the range from about 3.75 mm to about 4.45 mm, for example, 4.10 mm. In some embodiments, a longitudinal length 4078 between a center of second plurality of nodes 4044 and a center of fourth plurality of nodes 4075 is greater than each of longitudinal length 4076 and longitudinal length 4074. In some embodiments, longitudinal length 4078 is within the range from about 4.50 mm to about 5.10 mm, for example, about 4.80 mm.

In some embodiments such as the one shown in FIG. 4, nodes connecting adjacent struts can include a curved section defining a portion of the respective expandable cell. For example, each node of first plurality of nodes 4040 can include a first curved section 4070, and each node of second plurality of nodes 4044 can include a second curved section 4072. First and section curved sections 4070 and 4072 can each have a substantially constant radius. In some embodiments, first and second curved sections can have a radius within the range from about 0.03 mm to about 0.1 mm. For example, first and second curved sections can have a radius of about 0.06 mm.

In some embodiments, each node of first plurality of nodes 4040 can have a length within the range from about 0.1 mm to about 5 mm. In some embodiments, each node of second plurality of nodes 4044 can have a length within the range from about 0.4 mm to about 0.45 mm.

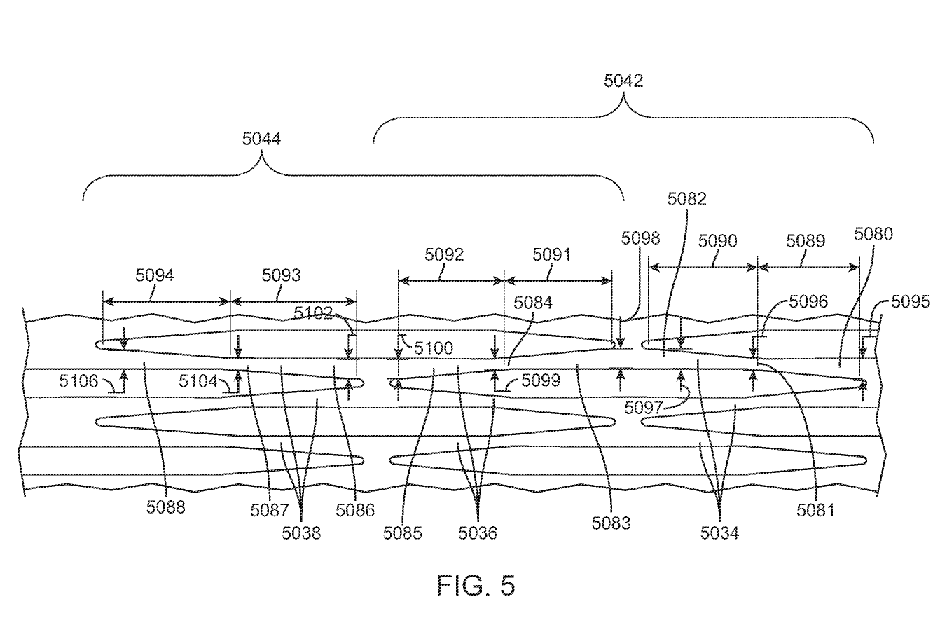

FIG. 5 illustrates an enlarged view of a first row 5042 and a second row 5046 of expandable cells of a frame in its original manufactured state—prior to being expanded or compressed—according to an embodiment. This cell pattern can be used, for example, in any section of frame 1000 illustrated in FIG. 1, frame 2000 illustrated in FIG. 2, or frame 3000 illustrated in FIG. 3. For example, this cell pattern can be used to form a portion of inflow section 2008, intermediate section 2010, or outflow section 2012 of frame 2000. And for example, this cell pattern can form the entire intermediate section 2010 of frame 2000. Similarly, this cell pattern can be used to form a portion of inflow section 3008, intermediate section 3010, or outflow section 3012 of frame 3000. In some embodiments, this cell pattern can form the entire intermediate section 3010 of frame 3000.

As shown in FIG. 5, struts of a first, second, or third set of struts 5034, 5036, and 5038 can be tapered. For example, each strut of first set of struts 5034 can include a proximal segment 5080, an intermediate segment 5081, and a distal segment 5082. Each strut of second set of struts 5036 can include a proximal segment 5083, an intermediate segment 5084, and a distal segment 5085. Each strut of third set of struts 5038 can include a proximal segment 5086, an intermediate segment 5087, and a distal segment 5088. In some embodiments, a width 5095 of proximal segment 5080 is greater than a width 5096 of intermediate segment 5081, and a width 5097 of distal segment 5082 is greater than width 5096 of intermediate segment 5081.

In some embodiments, width 5095 ranges from about 0.1 mm to about 1.2 mm. In some embodiments, width 5095 ranges from about 0.30 to about 0.36 mm, for example, about 0.33 mm. In some embodiments, width 5096 ranges from about 0.05 mm to about 1.0 mm. In some embodiments, width 5096 ranges from about 0.15 to about 0.21 mm, for example, about 0.18 mm. In some embodiments, width 5097 ranges from about 0.10 mm to about 1.2 mm. In some embodiments, width 5097 ranges from about 0.30 to about 0.36 mm, for example, about 0.33 mm.

In some embodiments, a width 5098 of proximal segment 5083 is greater than a width 5099 of intermediate segment 5084, and a width 5100 of distal segment 5085 is greater than width 5099. In some embodiments, width 5098 ranges from about 0.10 mm to about 1.2 mm. In some embodiments, width 5098 ranges from about 0.30 to about 0.36 mm, for example, about 0.33 mm. In some embodiments, width 5099 ranges from about 0.05 mm to about 1.0 mm. In some embodiments, width 5099 ranges from about 0.15 to about 0.21 mm, for example, about 0.18 mm. In some embodiments, width 5100 ranges from about 0.10 mm to about 1.2 mm. In some embodiments, width 5100 ranges from about 0.30 to about 0.36 mm, for example, about 0.33 mm.

In some embodiments, a width 5102 of proximal segment 5086 is greater than a width 5104 of intermediate segment 5087, and a width 5106 of distal segment 5088 is greater than width 5104. In some embodiments, width 5102 ranges from about 0.10 mm to about 1.2 mm. In some embodiments, width 5102 ranges from about 0.30 to about 0.36 mm, for example, about 0.33 mm. In some embodiments, width 5104 ranges from about 0.05 mm to about 1.0 mm. In some embodiments, width 5104 ranges from about 0.15 to about 0.21 mm, for example, about 0.18 mm. In some embodiments, width 5106 ranges from about 0.10 mm to about 1.2 mm. In some embodiments, width 5106 ranges from about 0.30 to about 0.36 mm, for example, about 0.33 mm.

In some embodiments, a length 5089 between a proximal end of proximal segment 5080 and a center of intermediate segment 5081 ranges from about 1.0 mm to about 2.5 mm. In some embodiments, length 5089 ranges from about 1.70 to about 1.62 mm, for example, about 1.66 mm. In some embodiments, a length 5090 between the center of intermediate segment 5081 and the distal end of distal segment 5082 ranges from about 1.0 to about 2.5 mm. In some embodiments, length 5090 ranges from about 1.74 to about 1.82 mm, for example, about 1.78 mm.

In some embodiments, a length 5091 between a proximal end of proximal segment 5083 and a center of intermediate segment 5084 ranges from about 1.0 to about 2.5 mm. In some embodiments, length 5091 ranges from about 1.72 to about 1.80 mm, for example, about 1.76 mm. In some embodiments, a length 5092 between the center of intermediate segment 5084 and the distal end of distal segment 5085 ranges from about 1.0 mm to about 2.5 mm. In some embodiments, length 5092 ranges from about 1.68 to about 1.76 mm, for example, about 1.72 mm.

In some embodiments, a length 5093 between a proximal end of proximal segment 5086 and a center of intermediate segment 5087 ranges from about 1.5 mm to about 3.0 mm. In some embodiments, length 5093 ranges from about 2.02 to about 2.10 mm, for example, about 2.06 mm. In some embodiments, a length 5094 between the center of intermediate segment 5087 and the distal end of distal segment 5088 ranges from about 1.5 mm to about 3.0 mm. In some embodiments, length 5094 ranges from about 2.04 to about 2.12 mm, for example, about 2.08 mm.

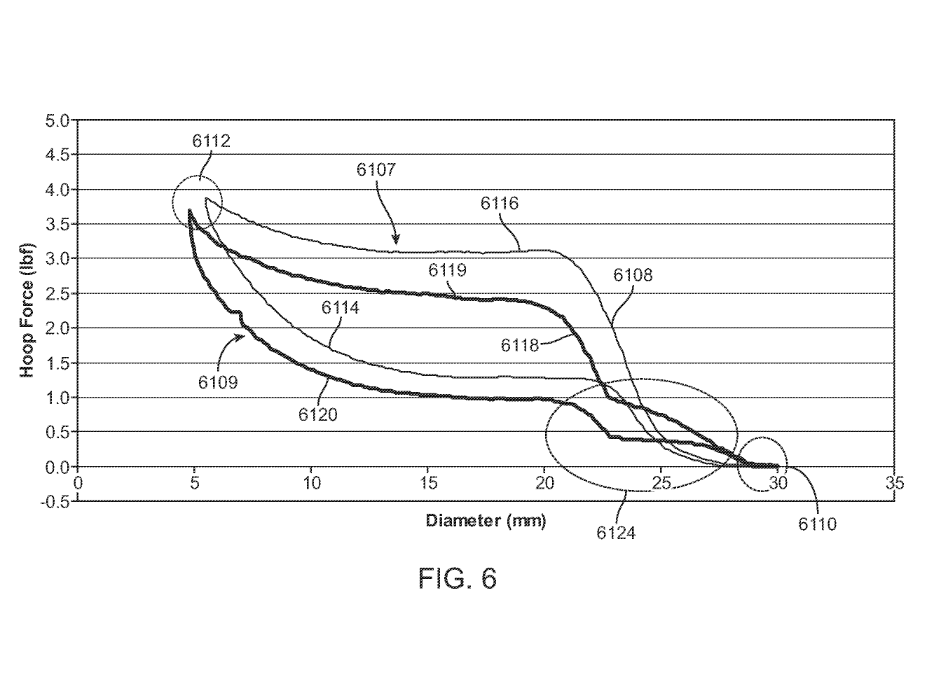

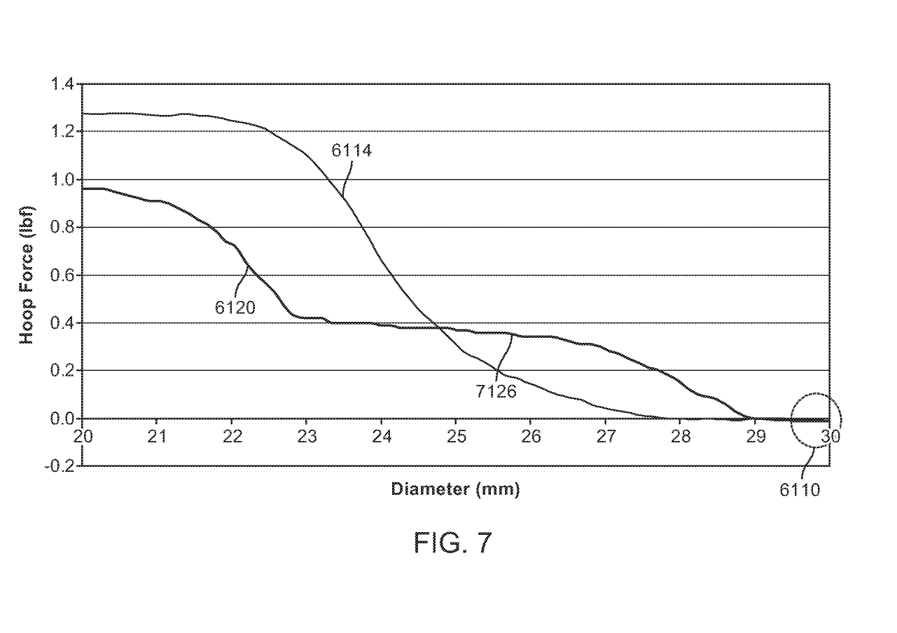

Tapered struts as described above with reference to FIGS. 4 and 5 can improve loading, deployment, and recapture performance in some embodiments. FIG. 6 illustrates a graph showing an exemplary loading and deployment force curve 6107 for a portion of a valve prosthesis frame, for example, an inflow section having straight struts and an exemplary loading and deployment force curve 6109 for a portion of a valve prosthesis frame, for example, an inflow section having tapered struts as described above with reference to FIGS. 4 and 5. FIG. 7 illustrates an enlarged view of a portion 6124 shown in FIG. 6.

As shown in FIG. 6, each force curve 6107 and 6109 forms a hysteresis loop. That is, force curve 6107 includes a loading portion 6108 and a deployment portion 6114. Loading portion 6108 of force curve 6107 represents the diameter of the prosthesis as it is compressed from an uncompressed, maximum diameter condition 6110 to a compressed, minimal diameter condition 6112. As the prosthesis is compressed, the hoop force, the radial outward force, generally increases. Loading portion 6108 can include a loading force plateau 6116 at which the magnitude of the hoop force, for example, about 3.1 lbf, remains substantially the same over a range of diameters, for example, from about 20 mm to about 12 mm. Deployment portion 6114 of force curve 6107 represents the diameter of the prosthesis as it is expanded from compressed, minimal diameter condition 6112 to the uncompressed, maximum diameter condition 6110. As the prosthesis is expanded, the hoop force generally decreases.

Force curve 6109 of a valve prosthesis having tapered struts as described above with reference to FIGS. 4 and 5 can comprise a loading portion 6118 and a deployment portion 6120. Loading portion 6118 represents the diameter of the prosthesis as it is compressed from uncompressed, maximum diameter condition 6110 to a compressed, minimal diameter condition 6112. As the prosthesis is compressed, the hoop force generally increases. Loading portion 6118 can include a loading force plateau 6119 at which the magnitude of the hoop force, for example, about 2.5 lbf, remains substantially the same over a range of diameters, for example, from about 19 mm to about 12 mm. Deployment portion 6120 represents the diameter of the prosthesis as it is expanded from the compressed, minimal diameter condition 6112 to the uncompressed, maximum diameter condition 6110. As the prosthesis is expanded, the hoop force generally decreases. As best seen in FIG. 7, deployment portion 6120 can include a deployment force plateau 7126 at which the magnitude of the hoop force, for example, about 0.4 lbf, remains substantially the same over a range of diameters, for example, from about 23 mm to about 27 mm.

A valve prosthesis having tapered struts as described above with reference to FIGS. 4 and 5 can be configured to lower the magnitude of the hoop force that occurs at a loading plateau relative to a similarly constructed valve prosthesis having straight struts. For example, as shown in FIG. 6, the magnitude of the hoop force that occurs at loading plateau 6119 of force curve 6109 of a valve prosthesis having tapered struts is less than the magnitude of the hoop force that occurs at loading plateau 6116 of force curve 6107 of a valve prosthesis having straight struts. A valve prosthesis can be more easily compressed for being loaded within a sheath or crimped on a catheter if the magnitude of hoop force at the loading plateau (for example, loading plateau 6116 or loading plateau 6119) is lowered. A valve prostheses can also be more easily recaptured within a sheath for repositioning if the magnitude of hoop force at the loading plateau (for example, loading plateau 6116 or loading plateau 6119) is lowered.

A valve prosthesis having tapered struts as described above with reference to FIGS. 4 and 5 can be configured such that deployment plateau 7126 occurs over a range of diameters. In some embodiments, the diameter range of deployment plateau 7126 generally corresponds to the intended patient annulus diameter range. In some embodiments, deployment plateau 7126 can span several millimeters. For example, deployment plateau 7126 can span for about 2 mm to about 10 mm. In some embodiments, deployment plateau 7126 can span about 4 mm. For example, as shown in FIG. 7, deployment plateau 7126 can start at a diameter of about 23 mm and end at a diameter of about 27 mm—a span of about 4 mm. In some embodiments, deployment plateau 7126 improves valve performance by providing a consistent outward radial force over the intended patient annulus range, which can help reduce paravalvular leakage, migration, and heart block.

A valve prosthesis having tapered struts, such as those described above with reference to FIG. 5, can be configured to minimize the area between the loading portion and the deployment portion of the loading and deployment force curve relative to a similarly constructed valve prosthesis having straight struts. For example, as shown in FIG. 6, the area between loading portion 6118 and deployment portion 6120 of force curve 6109 is smaller than the area between loading portion 6108 and deployment portion 6114 of force curve 6107. Minimizing the area between loading portion 6118 and deployment portion 6120 of force curve 6109 can minimize the loss of energy that may occur during loading an deployment.

Notably, force curves 6107 and 6108 are exemplary. Valve prostheses according to embodiments of the invention can have force curves that differ from the force curves 6107 and 6108 in FIGS. 6 and 7. In some embodiments, the uncompressed, maximum diameter condition 6110 can occur at a diameter other than about 30 mm. For example, the uncompressed, maximum diameter condition 6110 can occur at about 26 mm or at about 34 mm. In some embodiments, the compressed, minimum diameter condition 6112 can occur at a diameter other than about 5 to about 6 mm. For example, the compressed, minimal diameter condition 6112 can occur at about 3 mm or at about 9 mm. Similarly, the magnitudes of the hoop forces occurring at loading plateau 6119 and deployment plateau 7126 can be magnitudes other than about 2.5 lbf or about 0.4 lbf, respectively. For example, the magnitudes of the hoop forces occurring at loading plateau 6119 and deployment plateau 7126 can be about 1.5 lbf or about 0.2 lbf, respectively. Likewise, deployment plateau may begin at diameters of more than or less than about 23 mm and may end at diameters of more than or less than about 27 mm.

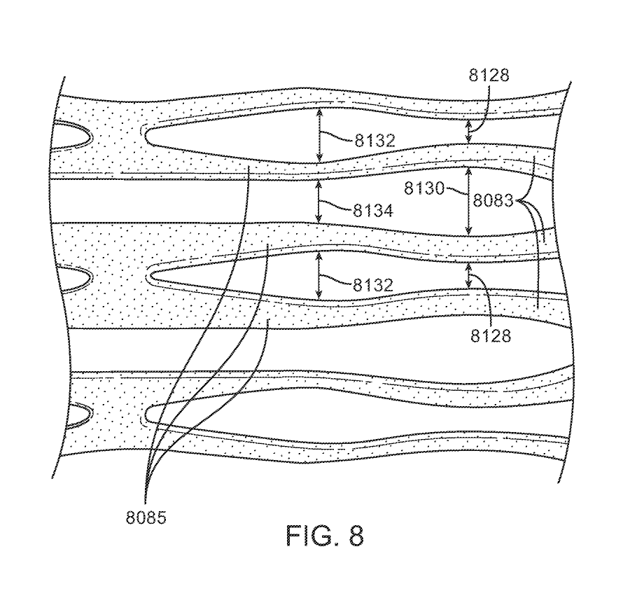

A valve prosthesis having tapered struts, such as those described above with reference to FIG. 5, can be configured to minimize tissue damage of the valve assembly when the valve prosthesis is in a compressed condition. In some embodiments, tissue damage is minimized by configuring the prosthesis frame such that when the frame is compressed there are gaps between circumferentially adjacent struts—the struts do not touch each other. In some embodiments, the gaps between circumferentially aligned strut segments alternate between small and large gaps. For example, FIG. 8 illustrates an enlarged of expandable cells of a frame in a compressed condition configured to minimize tissue damage according to an embodiment. As shown in FIG. 8, gaps formed between proximal segments 8083 of a set of struts, for example, first, second, or third sets of struts as described above with reference to FIG. 2 or 3, can circumferentially alternate between a small gap 8128 and a large gap 8130, and gaps formed between distal segments 8085 of the same set of struts can circumferentially alternate between a large gap 8132 and small gap 8134.

In some embodiments, the valve prosthesis can be configured such that the strut gaps compress tissue of the valve assembly no more than about 70 percent of twice the tissue thickness. For example, if the tissue has a thickness of about 0.30 mm, the valve prosthesis can be configured such that the strut gaps compress the tissue no more than about 0.18 mm (0.60 mm−(0.7*(2*0.30 mm))=0.18 mm).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A prosthesis comprising:
a collapsible and reexpandable frame, the frame having a longitudinal axis, the frame comprising:
first, second, and third sets of struts;
wherein the first and second sets of struts are connected at a first plurality of nodes to define a first row of expandable cells;
wherein the second and third sets of struts are connected at a second plurality of nodes to define a second row of expandable cells, the first row of expandable cells being proximal to the second row of expandable cells;
wherein each strut of the first set of struts comprises a first proximal segment with a first width, a first intermediate segment with a second width, and a first distal segment with a third width;
wherein each strut of the second set of struts comprises a second proximal segment with a fourth width, a second intermediate segment with a fifth width, and a second distal segment with a sixth width;
wherein each strut of the third set of struts comprises a third proximal segment with a seventh width, a third intermediate segment with an eighth width, and a third distal segment with a ninth width;
wherein the second width is smaller than each of the first and third widths;
wherein the fifth width is smaller than each of the fourth and sixth widths; and
wherein the eighth width is smaller than each of the seventh and ninth widths.

2. The prosthesis of claim 1, wherein:
the first width is about equal to the third width;
the fourth width is about equal to the sixth width; and
the seventh width is about equal to the ninth width.

3. The prosthesis of claim 1, wherein the second width, the fifth width, and the eighth width are about 42% to about 50% smaller than each of the first and third widths, each of the fourth and sixths widths, and each of the seventh and ninth widths, respectively.

4. The prosthesis of claim 1, wherein:
the first, third, fourth, sixth, seventh, and ninth widths each have a dimension within a range from about 0.1 mm to about 1.2 mm; and
the second, fifth, and eighth widths each have a dimension within a range from about 0.05 mm to about 1.0 mm.

5. The prosthesis of claim 1, wherein:
each of node of the first plurality of nodes comprises a first curved section having a first radius, the first curved section defining a portion of a respective cell of the second row of expandable cells, and the first radius ranging from about 0.03 mm to about 0.1 mm;
each of node of the second plurality of nodes comprises a second curved section having a second radius, the second curved section defining a portion of a respective cell of the first row of expandable cells, and the second radius ranging from about 0.03 mm to about 0.1 mm.

6. The prosthesis of claim 1, wherein:
each node of the first plurality of nodes having a tenth width, the tenth width ranging from about 0.1 mm to about 5 mm; and
each node of the second plurality of nodes having an eleventh width, the eleventh width ranging from about 0.1 mm to about 5 mm.

7. The prosthesis of claim 1, wherein:
the frame includes an outflow section, an intermediate section proximal to the outflow section, and an inflow section proximal to the intermediate section; and
the first and second rows of expandable cells form a portion of the intermediate section.

8. The prosthesis of claim 1, further comprising a valve body coupled to the frame.

9. A prosthesis comprising:
a collapsible and reexpandable frame, the frame having a longitudinal axis, the frame comprising:
an intermediate section; and
an inflow section that is proximal to the intermediate section, the inflow section comprising:
a concave saddle portion that is adjacent the intermediate section, and
an outwardly flared portion.

10. The prosthesis of claim 9, wherein the concave saddle portion comprises first and second sets of struts defining only a single row of expandable cells.

11. The prosthesis of claim 10, wherein the first set of struts are connected to the second set of struts at a plurality of nodes, wherein a first outer diameter of the frame at the plurality of nodes is smaller than a second outer diameter of the frame at a boundary between the saddle portion and the intermediate section, and wherein the first outer diameter is smaller than a third outer diameter at a boundary between the saddle portion and the outwardly flared portion of the inflow section.

12. The prosthesis of claim 9, wherein a contour of the concave saddle portion forms a smooth curve.

13. The prosthesis of claim 9, wherein the outwardly flared portion is convex, and wherein a contour of the outwardly flared portion section forms a smooth curve.

14. The prosthesis of claim 9, wherein a longitudinal length of the inflow section ranges from about 2.0 mm to about 50 mm.

15. The prosthesis of claim 9, wherein the frame further comprises an outflow section, the outflow section being distal to the intermediate section.

16. The prosthesis of claim 15, wherein the outflow section comprises a first portion that flares outwardly.

17. The prosthesis of claim 16, wherein the outflow section further comprises a second portion adjacent the intermediate section and proximal to the first portion of the outflow section, and wherein a contour of the second portion is substantially parallel with the longitudinal axis.

18. The prosthesis of claim 17, wherein:
the first portion of the outflow section comprises a plurality of V-shaped structures each comprising a first strut having a first end and a second end, and a second strut having a first end and a second end;
the second end of the first strut is connected to the second end of the adjacent second strut;
the first end of the first strut is connected to a first expandable cell;
the first end of the second strut is connected to a second expandable cell; and
a third expandable cell is between the first expandable cell and the second expandable cell.

19. The prosthesis of claim 16, further comprising a valve body;
wherein the valve body comprises a plurality of leaflets, adjoining leaflets being joined together at commissures; and
wherein the commissures are coupled to the first portion of the outflow section.

20. The prosthesis of claim 19, further comprising a valve body;
wherein the outflow section further comprises a second portion adjacent the intermediate section and proximal to the first portion of the outflow section;
wherein a contour of the second portion is substantially parallel with the longitudinal axis;
wherein the valve body comprises a plurality of leaflets, adjoining leaflets being joined together at commissures; and
wherein the commissures are coupled to the second portion of the outflow section.

21. The prosthesis of claim 16, wherein the first portion of the outflow section is convex.

22. The prosthesis of claim 9, further comprising a valve body;
wherein the frame has a longitudinal length;
wherein the valve body comprises a plurality of leaflets, adjoining leaflets being joined together at commissures; and
wherein the commissures are coupled to the outflow section such that a distance between the commissures and an outflow edge of the outflow section is about 20% to about 35% of the longitudinal length of the frame.
